(19) United States
(12) Patent Application Publication
(10) Pub. No.: US 2006/0183771 A1
(43) Pub. Date: Aug. 17, 2006

(54) Novel combination of selective factor VIIa and/or factor XIa inhibitors and selective plasma kallikrein inhibitors

(76) Inventors: Dietmar A. Seiffert, Boothwyn, PA (US); Robert M. Knabb, Avondale, PA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/354,777

(22) Filed: Feb. 15, 2006

Related U.S. Application Data
(60) Provisional application No. 60/653,831, filed on Feb. 17, 2005.

(51) Int. Cl. A61K 31/47

(52) U.S. Cl. 514/311

(57) ABSTRACT

The present invention relates to a novel pharmaceutical combination for treating thromboembolic and/or inflammatory diseases, wherein the combination has: (a) a first therapeutic agent independently selected from the group consisting of a selective Factor VIIa inhibitor, a selective Factor XIa inhibitor, a combination of the selective Factor VIIa and XIa inhibitors, or pharmaceutically acceptable salt forms thereof; and (b) a second therapeutic agent comprising a selective plasma kallikrein inhibitor or a pharmaceutically acceptable salt form thereof. The instant invention is also directed to a method and composition suitable for treating thromboembolic and/or inflammatory diseases using the novel combinations.

### RELATED APPLICATIONS

The application claims priority from provisional U.S. application Ser. No. 60/653,83 1, filed Feb. 17, 2005, incorporated herein by reference in its entirety.

### FIELD OF THE INVENTION

The present invention relates to novel combinations of selective Factor VIIa and/or Factor XIa inhibitors and selective plasma kallikrein inhibitors. The instant invention is also directed to methods and compositions suitable for treating thromboembolic and inflammatory diseases using the novel combinations.

### BACKGROUND OF THE INVENTION

While blood coagulation is a necessary and important part of the regulation of an organism's homeostasis, abnormal blood coagulation can also have deleterious effects. A thrombosis is the formation or presence of a blood clot, or thrombus, inside a blood vessel or cavity of the heart. Such a blood clot can lodge in a blood vessel, thereby blocking circulation and inducing a heart attack or stroke. Thromboembolic disorders, which are conditions that result from thrombosis, are one of the largest causes of mortality and disability in the industrialized world. Multiple links between thrombotic and inflammatory processes have been identified. This suggests that inflammation and thrombosis are closely coupled events.

Factor VII (FVII) and Factor XI (FXI) are generally believed to play essential roles in blood coagulation. The active form of Factor VII, designated as Factor VIIa (FVIIa), is involved in the extrinsic coagulation pathway. Walsh, P. N., Thromb. Haemostasis, 82, 234-242 (1999). Factor VIIa is a plasma serine protease involved in the initiation of the coagulation cascade. It binds with high affinity to tissue factor (TF) in the presence of calcium ions to form a complex with enhanced proteolytic activity. See Carson, S. D., and Brozna, J. P., Blood Coag. Fibrinol., 4, 281-292 (1993). Because of its key role in the coagulation cascade, researchers have postulated that inhibition of Factor VIIa could be used to treat or prevent thromboembolic disease. See Girard, T. J. & Nicholson, N. S., Curr. Opin. Pharmacol., 1, 159-163 (2001).

Work has accordingly been performed to identify and optimize Factor VIIa inhibitors. For example, U.S. Pat. No. 5,866,542 describes recombinant nematode anticoagulant proteins which inhibit Factor VIIa. U.S. Pat. No. 5,843,442 discloses monoclonal antibodies or antibody fragments possessing Factor VIIa inhibitory activity, and U.S. Pat. No. 5,023,236 presents tripeptides and tripeptide derivatives that inhibit Factor VIIa. Importantly, treatments that inhibit Factor VIIa activity appear to expose patients to less bleeding liabilities compared with mechanisms that target down-stream coagulation cascades, including Factors Xa and IIa (thrombin).

An alternative way to initiate coagulation occurs when blood is exposed to artificial surfaces, e.g., during hemodialysis, “on-pump” cardiovascular surgery, vessel grafts or bacterial sepsis. This process is termed “contact activation” and is part of the intrinsic coagulation pathway. Contact activation is a surface-mediated process responsible, in part, for the regulation of thrombosis and inflammation, and is mediated, at least in part, by fibrinolytic, complement, kininogen/kinin, and other humoral and cellular pathways. Contact activation leads to the generation of active Factor XII (XIIa) that in turn activates Factor XI to XIa. See Coleman, R., Contact Activation Pathway, Hemostasis and Thrombosis, 103-22 (Lippincott Williams & Wilkins 2001); and Schmaier A. H., Contact Activation, Thrombosis and Hemorrhage, 105-28 (1998).

The active form of Factor XI, Factor XIa, plays an important role in contact activation by initiating blood coagulation through activating Factor IX in the intrinsic coagulation pathway. Factor XI is a trypsin-like serine protease that is present in plasma at a relatively low concentration. Feedback activation of Factor XI by thrombin is believed to occur on negatively charged surfaces, most likely on the surface of activated platelets. Such platelets contain high affinity (0.8 nM) specific sites (130-500/platelet) for activated Factor XI. After activation, Factor XIa remains surface-bound and recognizes Factor IX as its normal macromolecular substrate. See Galiani, D., Trends Cardiovasc. Med. 2000, 10, 198-204. This feedback activation of Factor XI by thrombin can occur independently of the above-specified contact activation via Factor XIIa.

Genetic evidence indicates that Factor XI is not required for normal homeostasis, implying a superior safety profile of the Factor XI mechanism compared with competing antithrombotic mechanisms. See Gailani, D., Frontiers in Bioscience, 6, 201-207 (2001); and Gailani, D., et al., Blood Coagulation and Fibrinolysis, 8, 134-144 (1997). In contrast to hemophilia A (Factor VIII deficiency) or hemophilia B (Factor IX deficiency), mutations of the Factor XI gene causing Factor XI deficiency (hemophilia C) result in only a mild to moderate bleeding diathesis characterized primarily by postoperative or post-traumatic, but rarely spontaneous, hemorrhage. Taken together, these observations suggest that high levels of inhibition of Factor XIa should be well tolerated. These observations further point to the possibility of an increased therapeutic index of Factor XIa inhibitors.

Proteins or peptides that reportedly inhibit Factor XIa are disclosed in PCT Publication No. WO 01/27079. However, there are advantages to using small organic compounds in preparing pharmaceuticals because they generally have better oral biaavailability and compatibility in making formulations that aid in the delivery of the drug, as compared with large proteins or peptides. Small molecule inhibitors of Factor XIa are disclosed in PCT Publication Nos. WO 99/12935 and WO 02/42273.

Other studies have found a link between Factor XI activation and certain diseases. See, e.g., Minnema, M. C., et al., Arterioscler. Thromb. Vasc. Biol., 20, 2489-2493 (2000) (thrombin formation in acute myocardial infarction (AMI)); Murakami, T., et al., Arterioscler. Thromb. Vasc. Biol., 15, 1107-1113 (1995) (coronary arteriosclerosis); and Meijers, J. C. M., et al., N. Engl. J. Med., 342, 696-701 (2000) (venous thrombosis).

Plasma kallikrein, another protein involved in the intrinsic coagulation pathway, is a trypsin-like serine protease present in plasma at 35 to 50 ug/ml. The gene structure is similar to that of Factor XI in that, overall, the amino acid sequence of plasma kallikrein has 58% homology to Factor XI. Proteolytic activation of the zymogen plasma prekallikrein by Factor XIIa at an internal 1389-R390 bond yields a heavy chain (371 amino acids) and a light chain (248 amino acids). The active site of kallikrein is contained in the light chain. The light chain-of plasma kallikrein reacts with protease inhibitors, including alpha 2 macroglobulin and C1-inhibitor. Heparin has been found to significantly accelerate the inhibition of plasma kallikrein by antithrombin III in the presence of high molecular weight kininogen (HMWK). In blood, the majority of plasma kallikrein circulates in complex with HMWK. Kallikrein cleaves HMWK to liberate bradykinin. Bradykinin release results in the increase of vascular permeability and vasodilation. See Coleman, R., Contact Activation Pathway, Hemostasis and Thrombosis, 103-122 (Lippincott Williams & Wilkins 2001); and Schmaier A. H., Contact Activation, Thrombosis and Hemorrhage, 105-128 (1998). Genetic deficiencies of plasma kallikrein are well tolerated and do not result in a bleeding phenotype.

Plasma kallikrein is believed to be an important mediator of angioedema in patients with genetic deficiency of C1 esterase inhibitor (Hereditary Angioedema (HAE) patients). Williams, A., and Baird, L. G., DX-88 AND HAE: A Developmental Perspective, Transfusion and Apheresis Science, 29, 255-258 (2003); and Schapira, M., et al., Prekallikrein Activation and High-Molecular-Weight Kininogen Consumption in Hereditary Angioedema, New England Jour. Of Med., Vol. 308, No. 18, 1050-53 (1983).

It is desirable to find treatments for thromboembolic and/or inflammatory disorders with enhanced efficacy and lower bleeding liabilities. It is particularly desirable to find combinations of agents that allow for lower (i.e., sub-therapeutic and/or synergistic) dosages of each individual agent.

### SUMMARY OF THE INVENTION

The instant invention is for a novel pharmaceutical combination having: (a) a first therapeutic agent independently selected from the group consisting of a selective Factor VIIa inhibitor, a selective Factor XIa inhibitor, a combination of the selective Factor VIIa and XIa inhibitors, or pharmaceutically acceptable salt forms thereof; and (b) a second therapeutic agent comprising a selective plasma kallikrein inhibitor or a pharmaceutically acceptable salt form thereof.

It is preferred that the novel pharmaceutical combination further comprises: (c) a pharmaceutically acceptable carrier.

It is also preferred that the novel pharmaceutical combination further comprises: (d) a third therapeutic agent.

The present invention is also for a method of treating thromboembolic and/or inflammatory disorders by administering the novel pharmaceutical combination to a host in need of such treatment.

The instant invention is further directed to a pharmaceutical composition having the novel pharmaceutical combination.

Another aspect of the present invention provides a method for treating thromboembolic and/or inflammatory diseases, comprising administering to a host in need of such treatment the novel pharmaceutical combination of the present invention.

These and other objects will become apparent during the following detailed description.

### DETAILED DESCRIPTION OF THE INVENTION

Thus, in an embodiment, the present invention provides a novel method for treating a thromboembolic and/or inflammatory disorder, comprising: administering, to a host in need of such treatment, a therapeutically effective amount of:

(a) a first therapeutic agent independently selected from the group consisting of a selective Factor VIIa inhibitor, a selective Factor XIa inhibitor, a combination of the selective Factors VIIa and XIa inhibitors, or pharmaceutically acceptable salt forms thereof; and

(b) a second therapeutic agent comprising a selective plasma kallikrein inhibitor or a pharmaceutically acceptable salt form thereof.

In a preferred embodiment, at least one of the first and second therapeutic agents is administered in a sub-therapeutic dosage.

In another preferred embodiment, both the first and second therapeutic agents are administered in sub-therapeutic dosages.

In another preferred embodiment, the first and second therapeutic agents are administered simultaneously.

In another preferred embodiment, the first and second therapeutic agents are administered sequentially.

In another preferred embodiment, the present invention provides a novel method for treating a thromboembolic disorder, further comprising: administering, to a host in need of such treatment, a therapeutically effective amount of:

(c) a third therapeutic agent selected from other anti-coagulant or coagulation inhibitory agents, anti-platelet or platelet inhibitory agents, thrombin inhibitors, thrombolytic agents, fibrinolytic agents, anti-arrythmic agents, and cholesterol/lipid lowering agents.

In another preferred embodiment, the third therapeutic agent is selected from aspirin and pravastatin.

In another preferred embodiment, the third therapeutic agent is aspirin.

In another preferred embodiment, the third therapeutic agent is administered simultaneously with the first and second therapeutic agents.

In another preferred embodiment, the third therapeutic agent is administered in a sub-therapeutic dosage.

In another preferred embodiment, the throinboembolic disorder is selected from an arterial cardiovascular thromboembolic disorder, a venous cardiovascular thromboembolic disorder, an arterial cerebrovascular thromboembolic disorder, and a venous cerebrovascular thromboembolic disorder.

In another preferred embodiment, the thromboembolic disorder is selected from unstable angina, first inyocardial infarction, recurrent myocardial infarction, ischemic sudden death, transient ischemic attack, stroke, atherosclerosis, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis.

In another preferred embodiment, the thromboembolic disorder is selected from unstable angina, first myocardial infarction, recurrent myocardial infarction, ischemic sudden death, transient ischemic attack, and stroke.

In a further embodiment, the instant invention is for a novel pharmaceutical combination comprising the first and second therapeutic agents described hereinabove.

In another embodiment, the novel pharmaceutical combination further comprises: (c) a pharmaceutically acceptable carrier.

In yet another embodiment, at least one of the first and second therapeutic agents of the novel pharmaceutical combination is present in a sub-therapeutic dosage.

In a further embodiment, the first and second therapeutic agents of the novel pharmaceutical combination are present in sub-therapeutic dosages.

In still another embodiment, the present invention provides a novel pharmaceutical composition, comprising:

(a) a first therapeutic agent selected from the group consisting of a selective Factor VIIa inhibitor, a selective Factor XIa inhibitor, a combination of the selective Factor VIIa and XIa inhibitors, or a pharmaceutically acceptable salt form thereof;

(b) a second therapeutic agent comprising a selective plasma kallikrein inhibitor or a pharmaceutically acceptable salt form thereof; and

(c) a pharmaceutically acceptable carrier.

In another preferred embodiment, the present invention provides a novel pharmaceutical composition, further comprising:

(d) a third therapeutic agent selected from other anti-coagulant or coagulation inhibitory agents, anti-platelet or platelet inhibitory agents, thrombin inhibitors, thrombolytic agents, fibrinolytic agents, anti-arrythmic agents, and cholesterol/lipid lowering agents.

In another embodiment, the present invention provides a novel article of manufacture, comprising:

(a) a first container;

(b) a pharmaceutical composition located within the first container, wherein the pharmaceutical composition is as defined above; and

(c) a package insert stating that the pharmaceutical composition can be used for the treatment of a thromboembolic and/or inflammatory disorder.

In another preferred embodiment, the present invention provides a novel article of manufacture, further comprising:

(d) a second container, wherein components (a) and (b) are located within the second container and component (c) is located within or outside of the second container.

In another embodiment, the present invention provides a novel article of manufacture, comprising:

(a) a first container;

(b) a pharmaceutical composition located within the first container, wherein the composition is as defined above; and

(c) a package insert stating that the pharmaceutical composition can be used in combination with a third therapeutic agent to treat a thromboembolic and/or inflammatory disorder.

In another preferred embodiment, the present invention provides a novel article of manufacture, further comprising:

(d) a second container, wherein components (a) and (b) are located within the second container and component (c) is located within or outside of the second container.

The present invention may be embodied in other specific forms without departing from the spirit or essential attributes thereof. This invention encompasses all combinations of preferred aspects of the invention noted herein. It is understood that any and all embodiments of the present invention may be taken in conjunction with any other embodiment or embodiments to describe additional more preferred embodiments. It is also to be understood that each individual element of the preferred embodiments is intended to be taken individually as its own independent preferred embodiment. Furthermore, any element of an embodiment is meant to be combined with any and all other elements from any embodiment to describe an additional embodiment.

### DEFINITIONS

The phrases “first therapeutic agent,” “second therapeutic agent” and “third therapeutic agent” are referred to herein individually as “therapeutic agent” or “agent” and collectively as “therapeutic agents” or “agents.”

The phrase “pharmaceutically acceptable” is employed herein to refer to those therapeutic agents and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” and “pharmaceutically acceptable salt forms” refer to derivatives of the disclosed therapeutic agents wherein the therapeutic agent is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent combination, composition and/or compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, sulfanilic, 2-acetoxybenzoic, fumaric, toluenesulfonic, inethanesulfonic, ethane disulfonic, oxalic, isethionic, and the like.

The pharmaceutically acceptable salts of the present invention can be synthesized from the therapeutic agent that contains a basic or acidic moiety by conventional chemical methods. Generally, such salts can be prepared by reacting the free acid or base forms of these therapeutic agents with a stoichiometric amount of the appropriate base or acid in water or in an organic solvent, or in a mixture of the two; generally, non-aqueous media, like ether, ethyl acetate, ethanol, isopropanol or acetonitrile, are preferred. Lists of suitable salts are found in Remington's Pharmaceutical Sciences, 18th ed., 1445 (Mack Publishing Company, Easton, Pa., 1990), the disclosure of which is hereby incorporated by reference.

Since prodrugs are known to enhance numerous desirable qualities of pharmaceuticals (e.g., solubility, bioavailability, manufacturing, etc.), the therapeutic agents of the present invention may be delivered in prodrug form. Thus, the present invention is intended to cover prodrugs of the presently claimed therapeutic agents, methods of delivering the same and compositions and combinations containing the same. “Prodrugs” are intended to include any covalently bonded carriers that release an active parent drug of the present invention in vivo when such prodrug is administered to a mammalian subject. Prodrugs of the present invention are prepared by modifying functional groups present in the therapeutic agent in such a way that the modifications are cleaved, either in routine manipulation or in vivo, to the parent compound. Prodrugs include therapeutic agents of the present invention wherein a hydroxy, amino, or sulfhydryl group is bonded to any group that, when the prodrug of the present invention is administered to a mammalian subject, it cleaves to form a free hydroxyl, free amino, or free sulfhydryl group, respectively. Examples of prodrugs include, but are not limited to, acetate, formate, and benzoate derivatives of alcohol and amine functional groups in the therapeutic agents of the present invention.

As used herein, “treating” or “treatment” covers the treatment of a disease-state in a mammal, particularly in a human, and includes: (a) preventing the disease-state from occurring in a mammal, in particular, when such mammal is predisposed to the disease-state but has not yet been diagnosed as having it, which also includes reducing the risk of the disease-state occurring or reoccurring; (b) inhibiting the disease-state, i.e., arresting its development; and/or (c) relieving the disease-state, i.e., causing regression of the disease state.

“Therapeutically effective amount” is intended to mean an amount of therapeutic agents (a) and (b) of the present invention that is effective when administered in combination to treat a desired disease or condition. The combination of agents (a) and (b) is preferably a synergistic combination. “Synergy,” as described, for example, by Chou and Talalay, Adv. Enzyme Regul. 1984, 22:27-55, occurs when the effect of the compounds when administered in combination is greater than the additive effect of the compounds when administered alone as a single agent. In general, a synergistic effect is most clearly demonstrated at sub-optimal concentrations of the compounds (i.e., sub-therapeutic). Synergy can be in terms of lower cytotoxicity, increased antithrombotic effect, or some other beneficial effect of the combination compared with the individual components. When the combination of therapeutic agents (a), (b) and (c) is the desired embodiment, then the same shall have synergistic effects greater than the additive effect of the agents when administered alone as a single agent.

### Utility

The pharmaceutical combination of the present invention includes a first therapeutic agent and a second therapeutic agent. Exemplary first and second therapeutic agents are incorporated by reference herein as follows, although other such therapeutic agents are meant to be encompassed by the present invention.

U.S. Ser. No. 60/579,637 to Corte, et al., the entirety of which is incorporated by reference herein, provides compounds of Formula (I): 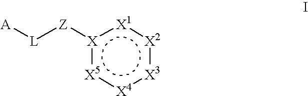
or a stereoisomer or pharmaceutically acceptable salt or solvate form thereof, wherein the variables A, L, Z, X, X¹, X², X³, X⁴, and X⁵ are as defined therein. The compounds of Formula (I) are useful as selective inhibitors of serine protease enzymes of the coagulation cascade and/or contact activation system; for example thrombin, factor Xa, factor XIa, factor IXa, factor VIIa and/or plasma kallikrein.

U.S. Ser. No. 60/579,638 to Corte, et al., the entirety of which is incorporated by reference herein, provides a method for treating a thrombotic or an inflammatory disorder by administering to a patient in need thereof a therapeutically effective amount of at least one compound of Formula (II): 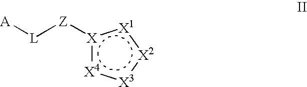
or a stereoisomer or pharmaceutically acceptable salt or solvate form thereof, wherein the variables A, L, Z, X, X¹, X², X³ and X⁴ are as defined therein. The compounds of Formula (II) are useful as selective inhibitors of serine protease enzymes of the coagulation cascade and/or contact activation system; for example thrombin, factor Xa, factor XIa, factor IXa, factor VIIa and/or plasma kallikrein. The invention of Formula (II) also provides compounds within the scope of Formula (II) and relates to pharmaceutical compositions comprising these compounds.

Publication No. US 2004/0235847A1 to Quan, et al., the entirety of which is incorporated by reference herein, provides 1,2,3,4-tetrahydroquinoline compounds of Formula (III): 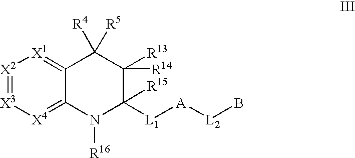
or a stereoisomer or pharmaceutically acceptable salt form thereof, wherein the variables A, B, L₁, L₂, X¹, X², X³, X⁴, R⁴, R⁵, R¹³, R¹⁴, R¹⁵ and R¹⁶ are as defined therein. The compounds of Formula (III) are useful as selective inhibitors of serine protease enzymes of the coagulation cascade and/or contact activation system; for example thrombin, factor Xa, factor XIa, factor IXa, factor VIIa and/or plasma kallikrein. This invention also relates to pharmaceutical compositions comprising the compounds of Formula (III) and methods of treating thromboembolic and/or inflammatory disorders using the same.

Publ. No. US 2004/0220206A1 to Smallheer, et al., the entirety of which is incorporated by reference herein, provides novel biarylmethyl indoline, indole and tetrahydroquinoline compounds of Formula (IV): 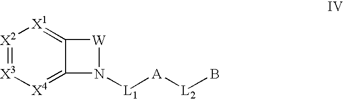
or a stereoisoiner or pharmaceutically acceptable salt or hydrate form thereof, wherein the variables A, B, L₁, L₂, X¹, X², X¹, X⁴ and W are as defined therein. The compounds of Formula (IV) are useful as selective inhibitors of serine protease enzymes of the coagulation cascade and/or contact activation system; for example thrombin, factor Xa, factor XIa, factor IXa, factor VIIa and/or plasma kallikrein. This invention also relates to pharmaceutical compositions comprising these compounds of Formula (IV) and methods of treating thromboembolic and/or inflammatory disorders using the same.

One preferred second therapeutic agent is DX-88, which is a potent and selective inhibitor of plasma kallikrein that has demonstrated a useful efficacy/safety ratio in the treatment of acute attacks of hereditary angioedema. Williams, A. and Baird, L. G., DX-88 AND HAE: A Developmental Perspective, Transfusion and Apheresis Science, 29, 255-258 (2003), the entirety of which is incorporated by reference herein. DX-88 may be obtained from Dyax Corporation, 300 Technology Square, Cambridge, Mass. 02139.

The efficacy of a combination of selective FVIIa/kallikrein and/or selective FXIa/kallikrein inhibitors in multiple thrombosis models and the reduction of bleeding liabilities suggest a possibility of replacing or reducing the amount of heparin used to treat throinboembolic disorders. A combined selective FVIIa/kallikrein and/or selective FXIa/kallikrein inhibitor may have the required profile (less bleeding liabilities due to improved therapeutic index; blocking, at least in part, inflammation by activating the classical component cascade through FXIIa). Selective FVIIa/kallikrein and/or selective Factor IXa/kallikrein inhibitors could serve dual functions, either as an add-on to current heparin therapy or as a replacement for heparin therapy, especially for persons known to be intolerant to heparin.

In another embodiment the present invention provides a method for modulation of the coagulation cascade and/or contact activation system comprising administering to a patient in need of such treatment a therapeutically effective amount of the pharmaceutical combination described above.

The instant pharmaceutical combination is useful as an anticoagulant for the treatment or prevention of thromboembolic disorders in mammals (i.e., Factor VIIa-, Factor XIa-, and/or kallikrein-associated disorders). In general, a thromboembolic disorder is a circulatory disease caused by blood clots (i.e., diseases involving platelet activation and/or platelet aggregation). The term “thromboembolic disorders” as used herein includes arterial cardiovascular thromboembolic disorders, venous cardiovascular thromboembolic disorders, arterial cerebrovascular thromboembolic disorders, and venous cerebrovascular thromboembolic disorders.

The tern “thromboembolic disorders” as used herein includes specific disorders selected from, but not limited to, unstable angina, first myocardial infarction, recurrent myocardial infarction, ischemic sudden death, transient ischemic attack, stroke, atherosclerosis, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis. It is noted that thrombosis includes occlusion (e.g., after a bypass) and reocclusion (e.g., during or after percutaneous transluminal coronary angioplasty).

In another embodiment, the present invention provides a method for treating inflammatory disorders comprising: administering to a patient in need of such treatment a therapeutically effective amount of the novel pharmaceutical combination disclosed in the present invention.

In another embodiment, the present invention provides a method wherein the inflammatory disorder is selected from the group consisting of sepsis, acute respiratory distress syndrome, and systemic inflammatory response syndrome.

In another embodiment, the present invention provides a novel method of treating a patient in need of thromboembolic disorder treatment, comprising: administering a compound of the present invention or a pharmaceutically acceptable salt or hydrate form thereof in an amount effective to treat a thromboembolic disorder.

In another embodiment, the present invention provides a method of treating a patient in need of inflammatory disorder treatment, comprising: administering a compound of the present invention or a pharmaceutically acceptable salt form thereof in an amount effective to treat an inflammatory disorder.

In another embodiment, the present invention provides a pharmaceutical combination further comprising a third therapeutic agent selected from one or more of potassium channel openers, calcium channel blockers, sodium hydrogen exchanger inhibitors, antiarrhythmic agents, antiatherosclerotic agents, anticoagulants, antithrombotic agents, prothrombolytic agents, fibrinogen antagonists, diuretics, antihypertensive agents, ATPase inhibitors, mineralocorticoid receptor antagonists, phospodiesterase inhibitors, antidiabetic agents, anti-inflammatory agents, antioxidants, angiogenesis modulators, antiosteoporosis agents, hormone replacement therapies, hormone receptor modulators, oral contraceptives, antiobesity agents, antidepressants, antianxiety agents, antipsychotic agents, antiproliferative agents, antitumor agents, antiulcer and gastroesophageal reflux disease agents, growth hormone agents and/or growth hormone secretagogues, thyroid mimetics, anti-infective agents, antiviral agents, antibacterial agents, antifungal agents, cholesterol/lipid lowering agents and lipid profile therapies, and agents that mimic ischemic preconditioning and/or myocardial stunning.

Administration of the first and second therapeutic agents preferably affords an efficacy advantage over the agents alone (i.e., a synergistic combination), preferably while permitting the use of lower doses of each (i.e., sub-therapeutic dosages). Further, administration of the first, second and/or third therapeutic agents, preferably affords an efficacy advantage over the agents alone (i.e., a synergistic combination), preferably while permitting the use of lower doses of each (i.e., sub-therapeutic dosages). A lower dosage minimizes the potential of side effects, thereby providing an increased margin of safety. It is preferred that at least one of the first and second therapeutic agents is administered in a sub-therapeutic dose. It is also preferred that both the first and second therapeutic agents be administered in sub-therapeutic dosages. It is even more preferred that the first, second, and third therapeutic agents be administered in sub-therapeutic dosages. As noted previously, sub-therapeutic is intended to mean an amount of a therapeutic agent that by itself does not give the desired therapeutic effect for the disease being treated. Synergistic combination is intended to mean that the observed effect of the combination is greater than the sum of the individual agents administered alone.

The first and second therapeutic agents may be administered at the same time or sequentially in any order at different points in time. Thus, each agent may be administered separately but sufficiently closely in time so as to provide the desired therapeutic effect. The first and second agents may also be formulated into a single pharmaceutical composition. The first, second, and third agents may also be administered at the same time or sequentially in any order at different points in time. The first, second, and third agents may also be formulated into a single pharmaceutical composition.

The therapeutic agents of the present invention are administered to a host in need thereof or are present in a pharmaceutical composition in a therapeutically effective amount. By “therapeutically effective amount” it is meant that appropriate amounts of the agents, when administered together or in combination with a third therapeutic agent, are effective to treat a thromboembolic and/or inflammatory disorder.

Additional therapeutic agents include other anti-coagulant or coagulation inhibitory agents, anti-platelet or platelet inhibitory agents, thrombin inhibitors, thrombolytic agents, fibrinolytic agents, anti-arrythmic agents, and cholesterol/lipid lowering agents.

Other anticoagulant agents (or coagulation inhibitory agents) that may be used in combination with the compounds of this invention include warfarin and heparin (either unfractionated heparin or any commercially available low molecular weight heparin), synthetic pentasaccharide, direct-acting thrombin inhibitors including hirudin and argatroban, as well as other selective Factor VIIa and Factor XIa inhibitors such as those described in the publications identified above.

The term anti-platelet agents (or platelet inhibitory agents), as used herein, denotes agents that inhibit platelet function, for example by inhibiting the aggregation, adhesion, or granular secretion of platelets. Agents include, but are not limited to, the various known non-steroidal anti-inflammatory drugs (NSAIDS) such as aspirin, ibuprofen, naproxen, sulindac, indomethacin, mefenamate, droxicam, diclofenac, sulfinpyrazone, piroxicam, and pharmaceutically acceptable salts or prodrugs thereof. Of the NSAIDS, aspirin (acetylsalicyclic acid or ASA) and piroxicam are preferred. Other suitable platelet inhibitory agents include IIb/IIIa antagonists (e.g., tirofiban, eptifibatide, and abciximab), thromboxane-A2-receptor antagonists (e.g., ifetroban), thromboxane-A2-synthetase inhibitors, PDE-III inhibitors (e.g., dipyridamole), and pharmaceutically acceptable salts or prodrugs thereof. 100731 The term thrombin inhibitors (or anti-thrombin agents), as used herein, denotes inhibitors of the serine protease thrombin. By inhibiting thrombin, various thrombin-mediated processes, such as thrombin-mediated platelet activation (that is, for example, the aggregation of platelets and/or the granular secretion of plasminogen activator inhibitor-I and/or serotonin) and/or fibrin formation, are disrupted. A number of thrombin inhibitors are known to one of skill in the art and these inhibitors are contemplated to be used in combination with the present compounds. Such inhibitors include, but are not limited to, boroarginine derivatives, boropeptides, heparins, hirudin, argatroban, and melagatran, including pharmaceutically acceptable salts and prodrugs thereof. Boroarginine derivatives and boropeptides include N-acetyl and peptide derivatives of boronic acid, such as C-terminal α-aminoboronic acid derivatives of lysine, ornithine, arginine, homoarginine, and corresponding isothiouronium analogs thereof. The term hirudin, as used herein, includes suitable derivatives or analogs of hirudin, referred to herein as hirulogs, such as disulfatohirudin.

The tern thrombolytics or fibrinolytic agents (or thrombolytics or fibrinolytics), as used herein, denotes agents that lyse blood clots (thrombi). Such agents include tissue plasminogen activator (natural or recombinant) and modified forms thereof, anistreplase, urokinase, streptokinase, tenecteplase (TNK), lanoteplase (nPA), Factor VIIa inhibitors, PAI-1 inhibitors (i.e., inactivators of tissue plasminogen activator inhibitors), alpha2-antiplasmin inhibitors, and anisoylated plasminogen streptokinase activator complex, including pharmaceutically acceptable salts or prodrugs thereof. The term anistreplase, as used herein, refers to anisoylated plasminogen streptokinase activator complex, as described, for example, in EP 028,489, the disclosure of which is incorporated herein by reference. The term urokinase, as used herein, is intended to denote both dual and single chain urokinase, the latter also being referred to herein as prourokinase.

Examples of suitable anti-arrythmic agents for use in combination with the present compounds include: Class I agents (such as propafenone); Class II agents (such as carvadiol and propranolol); Class III agents (such as sotalol, dofetilide, amiodarone, azimilide, and ibutilide); Class IV agents (such as ditiazem and verapamil); K⁺ channel openers such as IAch inhibitors, and IKur inhibitors (e.g., compounds such as those disclosed in WO01/40231).

Examples of suitable cholesterol/lipid lowering agents and lipid profile therapies for use in combination with the compounds of the present invention include: HMG-CoA reductase inhibitors (e.g., pravastatin, lovastatin, atorvastatin, simvastatin, NK-104 (a.k.a. itavastatin, or nisvastatin or nisbastatin), and ZD-4522 (a.k.a. rosuvastatin, atavastatin, or visastatin)); squalene synthetase inhibitors; fibrates; bile acid sequestrants (such as questran); ACAT inhibitors; MTP inhibitors; lipooxygenase inhibitors; cholesterol absorption inhibitors; and cholesterol ester transfer protein inhibitors (e.g., CP-529414).

The present invention also encompasses an article of manufacture. As used herein, article of manufacture is intended to include, but not be limited to, kits and packages. The article of manufacture of the present invention comprises: (a) a first container; (b) a pharmaceutical composition located within the first container, wherein the pharmaceutical composition comprises: a first and second therapeutic agent or pharmaceutically acceptable salt forms thereof, as described hereinabove; and (c) a package insert stating that the pharmaceutical composition can be used for the treatment of a thromboembolic and/or inflammatory disorder (as defined previously). In another embodiment, the package insert states that the pharmaceutical composition can be used in combination (as defined previously) with a third therapeutic agent to treat a thromboembolic and/or inflammatory disorder. The article of manufacture can further comprise: (d) a second container, wherein components (a) and (b) are located within the second container and component (c) is located within or outside of the second container. Located within the first and second containers means that the respective container holds the item within its boundaries.

The first container is a receptacle used to hold a pharmaceutical composition. This container can be for manufacturing, storing, shipping, and/or individual/bulk selling. This first container is intended to cover a bottle, jar, vial, flask, syringe, tube (e.g., for a cream preparation), or any other container used to manufacture, hold, store, or distribute a pharmaceutical product.

The second container is one used to hold the first container and, optionally, the package insert. Examples of the second container include, but are not limited to, boxes (e.g., cardboard or plastic), crates, cartons, bags (e.g., paper or plastic), pouches, and sacks. The package insert can be physically attached to the outside of the first container via tape, glue, staple, or another method of attachment, or it can rest inside the second container without any physical means of attachment to the first container. Alternatively, the package insert can be located on the outside of the second container. When located on the outside of the second container, it is preferable that the package insert is physically attached via tape, glue, staple, or another method of attachment. Alternatively, it can be adjacent to or touching the outside of the second container without being physically attached.

The package insert is a label, tag, marker, etc., that recites information relating to the pharmaceutical composition located within the first container. The information recited will usually be determined by the regulatory agency governing the area in which the article of manufacture is to be sold (e.g., the United States Food and Drug Administration). Preferably, the package insert specifically recites the indications for which the pharmaceutical composition has been approved. The package insert may be made of any material on which a person can read information contained therein or thereon. Preferably, the package insert is a printable material (e.g., paper, plastic, cardboard, foil, adhesive-backed paper or plastic, etc.) on which the desired information has been formed (e.g., printed or applied).

### Dosage and Formulation

The compounds of this invention can be administered in such oral dosage forms as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular form, all using dosage forms well known to those of ordinary skill in the pharmaceutical arts. They can be administered alone, but generally will be administered with a pharmaceutical carrier selected on the basis of the chosen route of administration and standard pharmaceutical practice.

The dosage regimen for the compounds of the present invention will, of course, vary depending upon known factors, such as the pharmacodynamic characteristics of the particular agent and its mode and route of administration; the species, age, sex, health, medical condition and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to prevent, counter or arrest the progress of the thromboembolic and/or inflammatory disorder.

By way of general guidance, the daily oral dosage of each active ingredient, when used for the indicated effects, will range between about 0.001 to 1000 mg/kg of body weight, preferably between about 0.01 to 100 mg/kg of body weight per day, and most preferably between about 1.0 to 20 mg/kg/day. Intravenously, the most preferred doses will range from about 1 to about 10 mg/kg/minute during a constant rate infusion. Compounds of this invention may be administered in a single daily dose, or the total daily dosage may be administered in divided doses of two, three, or four times daily.

Compounds of this invention can be administered in intranasal form via topical use of suitable intranasal vehicles, or via transdermal routes, using transdermal skin patches. When administered in the form of a transdermal delivery system, the dosage administration will, of course, be continuous rather than intermittent throughout the dosage regimen.

The compounds are typically administered in admixture with suitable pharmaceutical diluents, excipients or carriers (collectively referred to herein as “pharmaceutically acceptable carriers”) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Compounds of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamiidephenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolyiners of hydrogels.

Dosage forms (pharmaceutical compositions) suitable for administration may contain from about I milligram to about 100 milligrams of active ingredient per dosage unit. In these pharmaceutical compositions the active ingredient will ordinarily be present in an amount of about 0.5-95% by weight based on the total weight of the composition.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar-coated or film-coated to mask any unpleasant taste and protect the tablet from the atmosphere, or enteric-coated for selective disintegration in the gastrointestinal tract.

Liquid dosage forms for oral administration can contain coloring and flavoring to increase patient acceptance.

In general, water, a suitable oil, saline, aqueous dextrose (glucose), and related sugar solutions and glycols, such as propylene glycol or polyethylene glycols, are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl-or propyl-paraben, and chlorobutanol.

Suitable pharmaceutical carriers are described in Remington's Pharmaceutical Sciences, Mack Publishing Company, a standard reference text in this field.

These as well as other ways of minimizing contact between the components of combination products of the present invention, whether administered in a single dosage form or administered in separate forms but at the same time by the same manner, will be readily apparent to those skilled in the art, once armed with the present disclosure.

The invention having been thus described, it will be apparent that the same may be varied in many ways without departing from the spirit and scope of the invention, as defined by the following claims.

What is claimed is:

1. A pharmaceutical combination, comprising:
(a) a first therapeutic agent independently selected from the group consisting of a selective Factor VIIa inhibitor, a selective Factor XIa inhibitor, a combination of the selective Factors VIIa and XIa inhibitors, or pharmaceutically acceptable salt forms thereof; and
(b) a second therapeutic agent comprising a selective plasma kallikrein inhibitor or a pharmaceutically acceptable salt form thereof.

2. The pharmaceutical combination of claim 1, further comprising: (c) a pharmaceutically acceptable carrier.

3. The pharmaceutical combination of claim 1, wherein at least one of the first and second therapeutic agents is present in a sub-therapeutic dosage.

4. The pharmaceutical combination of claim 1, wherein both of the first and second therapeutic agents are present in sub-therapeutic dosages.

5. The pharmaceutical combination of claim 1, wherein the second therapeutic agent comprises DX-88.

6. A method for treating a thromboembolic and/or inflammatory disorder, comprising administering the pharmaceutical combination of claim 1 to a host in need of such treatment.

7. The method of claim 6 wherein said disorder comprises a disorder associated with a mechanism involved with at least one of a contact activation system, a kallikrein/kinin system and/or an extrinsic coagulation system.

8. The method of claim 7 wherein said disorder activated by the contact activation system comprises a disorder activated by a contact activation system by artificial surfaces.

9. The method of claim 8 wherein said disorder activated by the contact activation system by artificial surfaces is selected from coronary artery bypass grafting (GABG), hemodialysis, on-pump cardiovascular surgery, vessel graft, bacterial sepsis, septic shock and renal dialysis.

10. The method of claim 7 wherein said disorder activated by the contact activation system comprises a disorder deficient of naturally occurring inhibitors of the contact activation system.

11. The method of claim 10 wherein said disorder deficient of naturally occurring inhibitors of the contact activation system comprises hereditary angiodema (HAE), thrombosis, disseminated intravascular coagulation (DIC) and adult respiratory distress syndrome (ARDS).

12. The pharmaceutical combination of claim 1, further comprising: (d) a third therapeutic agent.

13. The pharmaceutical combination of claim 12, wherein the third therapeutic agent is selected from as least one of other anti-coagulant or coagulation inhibitory agents, anti-platelet or platelet inhibitory agents, thrombin inhibitors, thrombolytic agents, fibrinolytic agents, anti-arrythmic agents, and cholesterol/lipid lowering agents.

14. A pharmaceutical composition, comprising:
(a) a first therapeutic agent selected from the group consisting of a selective Factor VIIa inhibitor, a selective Factor XIa inhibitor, a combination of the selective Factors VIIa and XIa inhibitors, or a pharmaceutically acceptable salt form-thereof; thereof;
(b) a second therapeutic agent comprising a selective plasma kallikrein inhibitor or a pharmaceutically acceptable salt form thereof; and
(c) a pharmaceutically acceptable carrier.

15. The pharmaceutical composition of claim 14, further comprising:
(d) a third therapeutic agent selected from at least one of other anti-coagulant or coagulation inhibitory agents, anti-platelet or platelet inhibitory agents, thrombin inhibitors, throinbolytic agents, fibrinolytic agents, anti-arrythmic agents, and cholesterol/lipid lowering agents.
